SEQUENCE LISTING (14 sequences)

SEQ ID NO: 1 - RNA, 33 nt, Artificial Sequence
  Aptamer
ggcgauaccagccgaaaggcccuuggcagcguc

SEQ ID NO: 2 - DNA, 30 bp, Artificial Sequence
  Aptamer
gacaaggaaaatccttcaatgaagtgggtc

SEQ ID NO: 3 - DNA, 29 bp, Artificial Sequence
  Aptamer
cacctgggggagtattgcggaggaaggtt

SEQ ID NO: 4 - DNA, 30 bp, Artificial Sequence
  Aptamer
ggaagagatggcgactaaaacgacttgtcg

SEQ ID NO: 5 - RNA, 44 nt, Artificial Sequence
  Aptamer
gacgagaaggagugcugguucuacuagcgguuaggucacucguc

SEQ ID NO: 6 - DNA, 60 bp, Artificial Sequence
  Aptamer
gggatcgaaacgtagcgccttcgatcccgcatgaccagggcaaacggtaggtgagtggtc

SEQ ID NO: 7 - RNA, 57 nt, Artificial Sequence
  Aptamer
gggugacuugguuuagguaaugagucacccgggacgagguuuagcuacacucguccc

SEQ ID NO: 8 - DNA, 38 bp, Artificial Sequence
  Aptamer
acgaggggacgaaagtccccggacaatcagacacggtc

SEQ ID NO: 9 - protein, 19 aa, Artificial Sequence
  cell penetrating peptide
CKLALKLALKALKAALKLA

SEQ ID NO: 10 - RNA, 40 nt, Artificial Sequence
  Aptamer
ucaucggaggagcgguguggugaggugacgcggaaaaggu

SEQ ID NO: 11 - DNA, 44 bp, Artificial Sequence
  Aptamer
gtagtaagaactaaagtaaaaaaaaaattaaagtagccacgctt

SEQ ID NO: 12 - DNA, 44 bp, Artificial Sequence; identical to SEQ ID NO: 11
  Aptamer

SEQ ID NO: 13 - DNA, 43 bp, Artificial Sequence
  Aptamer
attgaggtcatagaaaggaaaaagaataaaattaaccacacgg

SEQ ID NO: 14 - DNA, 45 bp, Artificial Sequence
  Aptamer
atagccaacatatggcagcatacctatagtaagaactcgattttt

(19) United States
(12) Patent Application Publication
(10) Pub. No.: US 2012/0129725 A1
(43) Pub. Date: May 24, 2012

(54) NUCLEIC ACID NANO-BIOSENSORS

(75) Inventors: Lars Folke Olsen, Hesselager (DK); Cengiz Oezalp, Odense M (DK); Lise Junker Nielsen, Odense M (DK)

(73) Assignee: Syddansk Universitet, Odense M (DK)

(21) Appl. No.: 13/382,113

(22) PCT Filed: Jul. 7, 2010
(86) PCT No.: PCT/DK10/50178

§ 371 Date: Feb. 7, 2012

Related U.S. Application Data
(60) Provisional application No. 61/224,487, filed on Jul. 10, 2009.
(60) Provisional application No. 61/329,161, filed on Apr. 29, 2010.

(30) Foreign Application Priority Data
Jul. 10, 2009 (DK) PA 2009 00852

(51) Int. Cl. C40B 30/04; C12Q 1/68; G01N 33/566; B82Y 15/00

(52) U.S. Cl. 506/9; 436/501; 435/6.1; 977/773; 977/920

(57) ABSTRACT

There is provided nanobiosensors and more particularly sensors comprising one or more aptamers or other functional nucleic acids adapted for signalling incorporated within a nanoparticle comprising polyacrylamide or other suitable polymer. Moreover, there is provided a novel DNA aptamer, which selectively binds to ATP. There is also provided a novel nanobiosensor for monitoring ATP concentrations in samples, including biological samples; this new approach may be used to monitor kinase activity in a given sample.

### FIELD OF THE INVENTION

The invention relates generally to nanobiosensors and more particularly to sensors comprising one or more aptamers or other functional nucleic acids adapted for signalling incorporated within a nanoparticle comprising polyacrylamide or other suitable polymer. Moreover, the present invention provides a novel DNA aptamer, which selectively binds to ATP. The present invention also provides a novel nanobiosensor for monitoring ATP concentrations in samples, including biological samples; this new approach may be used to monitor kinase activity in a given sample.

### BACKGROUND

Aptamers are single-stranded oligonucleotides, including both RNAs and DNAs, that express high binding selectivity and affinity for a wide variety of biological, organic or inorganic molecules. Often referred to as “chemical antibodies”, aptamers typically exhibit comparable affinity and greater selectivity for specific “target ligands” than can be achieved by monoclonal protein antibodies.

The high affinity and selectivity of aptamers for their “targets” derives from their ability to fold into distinct conformations. Aptamers for a given “target” are selected using an in vitro selection process termed SELEX (systematic evolution of ligands by exponential enrichment). Typically, a library of DNA or RNA transcribed from a DNA library is exposed to a column having a specific ligand immobilized on an inert matrix. Libraries of synthetic sequences are typically used, including “partially engineered” sequences which contain random sequence flanking sequences known to form simple secondary structures such as stems, tetraloops, pseudoknots or hairpins. Olignocleotides that bind the ligand of interest are then eluted and amplified by PCR. This process is repeated sequentially until highly specific oligonucleotides predominate. These are then sequenced and identified as aptamers.

Because of the essentially infinite range of possible oligonucleotide sequences, having correspondingly wide molecular diversity, aptamers can be isolated that have high affinity for virtually any molecule. High affinity aptamers have already been reported for a wide variety of molecules, including organic dyes, D- and L-amino acids, antibiotics, peptides, proteins, vitamins, drugs, metal ions, nucleotide triphosphates and even whole cells.

Aptamers have been used previously as recognition elements for analytical devices. For review, see [1, 2, 3, 4 and 5] and for recent examples see [6, 7, 8 and 9]. Several methods have also been reported for modifying aptamers so as to provide “real-time” fluorescence signalling capabilities. For review see [10 and 11]. Typically fluorophores or fluorophore/quench pairs are attached at a conformationally sensitive position of an aptamer. A conformational change induced by ligand binding induces a corresponding detectable change in fluorescence signal intensity. A particularly promising approach to “signalling aptamers” has relied upon attachment of “molecular beacons”. These are oligonucleotide sequences that typically fold into a hairpin-shape, with an internally quenched fluorophore. Binding of a target nucleotide sequence restores fluorescence and thereby provides real-time fluorescence signalling of the binding event. Adapted “molecular beacons” can be attached to aptamers to provide fluorescence signalling of aptamer-target binding.

Considerable interest has arisen in intracellular, in vivo applications of “signalling aptamers”. These could prove useful in intracellular imaging of gene expression, metabolite production or other metabolic processes. Aptamers have been successfully internalized in vivo by conjugation to a variety of cellular-uptake systems, including cellular uptake proteins and to aptamers engineered to provide cell-specific intracellular delivery. See e.g. [12].

The utility of “signalling aptamers” in vivo has been limited by their general susceptibility to nuclease digestion. Aptamers are often chemically modified at the 3′ and 5′ ends, or “capped”, to provide resistance to cellular exonucleases. Resistance to endonucleases has required much more elaborate approaches, including modified SELEX processes using libraries of metabolically stable “mirror image” nucleotides or other artificial, chemically modified, nuclease-resistant nucleotides. See e.g. [13].

Here the inventors report that, surprisingly, “signalling aptamers” suitable for real-time intracellular imaging in vivo retain full functionality after incorporation within a porous, polyacrylamide nanoparticle having average particle size<50 nm. This comparatively simple, non-covalent modification provides aptamer nanobiosensors which are stabilized against nuclease digestion, without requirement for chemically modified nucleotides.

In addition to aptamers, other functional nucleic acids, such as nucleic acid enzymes and combinations of aptamers and nucleic acid enzymes, may also be adapted for signalling and incorporated within a nanoparticle.

Incorporation within a nanoparticle extends aptamer shelf-life and also provides a solid affinity matrix that can be used in affinity chromatography applications.

The nucleic acid nanobiosensors can also be used in cell-free systems to detect target concentrations in sample fluids.

Many cellular processes depend on ATP, e.g. ion transport across membranes, cell motility and biosynthetic reactions [44]. In cell-signaling cascades protein kinases transfer a phosphate group from ATP to key regulatory proteins that serve in the control of cell metabolism, growth and differentiation [45]. Thus, assays to monitor ATP concentration in both bioassays and in cellular environments have wide applications in biochemical and biomedical research. Despite the important role played by ATP in biological systems, there exist only few sensors which can monitor ATP in real-time and those have significant limitations in intracellular usage. In most ATP-utilizing metabolic reactions, ATP is converted to ADP (adenosine-5′-diphosphate) and only to a lesser extent to AMP (adenosine-5′-monophosphate). ADP is recycled to ATP through phosphorylation reactions. Therefore, the challenge in developing a specific ATP sensor is to produce one which can differentiate between ATP and ADP. However, only a few such sensors have been reported.

Furthermore, most of the available sensors suffer from the fact that they have a high affinity for ATP and therefore will only have a limited use in many bioassays and in cellular environments which have ATP concentrations in the millimolar range. Recently, three different kinds of biosensor were reported for real-time monitoring of ATP: (i) A sandwich stacking of pyrene-adenine-pyrene was designed to measure ATP concentrations in HeLa cells [46]; (ii) a genetically-encoded biosensor for ADP was developed by engineering the bacterial actin ParM to measure ATPase and kinase activity [47]; (iii) finally, the ε-subunit of F0F1-ATP synthase was sandwiched between two fluorescent proteins to develop a genetically-encoded Förster resonance energy transfer (FRET) type indicator of ATP [48].

An alternative approach is to develop a functional aptamer sensor for a simple and accurate real-time ATP detection in bioassays and in cellular environments. Aptamers are single stranded nucleic acids with specific affinity for their targets. The in vitro selection methodology can provide an aptamer for almost any kind of target with pre-chosen affinity and thus may be considered as an attractive diagnostic and sensing molecule. Moreover, once an aptamer has been selected, it can be chemically synthesized and used directly as a probe unlike genetically-coded sensors, which often require molecular cloning and cellular expression procedures.

RNA [49] and DNA [50] aptamers targeting ATP were selected in the early years after the discovery of aptamers. Most aptamer-based sensor designs were tested by using almost exclusively either the DNA aptamer for ATP or the thrombin-binding aptamer as model systems for proof-of-concept studies [51]. DNA aptamers were preferred mainly because DNA is easier to handle as it is not easily degraded by nucleases. Both aptamers are selective for ATP compared to other nucleotide triphosphates, but have only limited selectivity for ATP compared to other adenine-nucleotides (ADP, AMP). The issue of selection on the phosphate moiety has later been addressed by selecting a second RNA aptamer by applying a selective pressure for the triphosphate group of the molecule [52].

### SUMMARY OF THE INVENTION

Ligand-selective aptamer sequences comprising signalling functionalities can be incorporated within a porous polymer nanoparticle with essentially full activity. This provides a nuclease-resistant nanobiosensor, typically with average particle sizes<50 nm diameter. Other functional nucleic acids such as nucleic acid enzymes may also be adapted for signalling and incorporated within a nanoparticle. The nanobiosensors can be incorporated within living cells, provide real-time intracellular imaging and measurements in vivo, and can also be used for cell-free analysis of fluid samples and as affinity chromatography media.

In a first aspect the present invention provides a nanobiosensor comprising an aptamer sequence incorporated within a polymer nanoparticle having average particle size<100 nm. Preferably, the aptamer was selected from a library of modified nucleotides and comprises a fluorescence/quench pair. In a preferred embodiment of the present invention the polymer is polyacrylamide. In a particularly preferred embodiment the aptamer is a DNA sequence. Preferably the aptamer is any one of SEQ ID NO. 1-8, 10 or 11-14.

In another aspect the present invention provides a nanobiosensor comprising an aptamer sequence incorporated within a polyacrylamide nanoparticle having average particle size<50 nm, wherein the aptamer sequence is either
modified at the 3′ end with a PEG linker, a short hybridizing sequence complementary to the 5′ end of the aptamer sequence and one partner of a fluorescence/quench pair, and
modified at the 5′ end with the other partner of a fluorescence/quench pair
or
modified at the 5′ end with a PEG linker, a short hybridizing sequence complementary to the 3′ end of the aptamer sequence and one partner of a fluorescence/quench pair, and
modified at the 3′ end with the other partner of a fluorescence/quench pair.

In a third aspect the present invention provides a nanobiosensor comprising an aptamer sequence incorporated within a polyacrylamide nanoparticle having average particle size<50 nm, wherein the aptamer sequence is either
modified at the 3′ end with an extender sequence and one partner of a fluorescence/quench pair, and
modified at the 5′ end with a complementary extender sequence and the other partner of a fluorescence/quench pair,
or
modified at the 5′ end with an extender sequence and one partner of a fluorescence/quench pair, and
modified at the 3′ end with a complementary extender sequence and the other partner of a fluorescence/quench pair.

In a fourth aspect the present invention provides a novel DNA aptamer selected from the group consisting of SEQ. ID NO 11, SEQ. ID NO 12, SEQ. ID NO 13, SEQ. ID NO 14.

Preferably, the DNA aptamer of claim 24, wherein the aptamer sequence is modified by a molecular beacon structure.

### BRIEF DESCRIPTION OF THE DRAWINGS

FIG. 1. ATP binding to ATP-selective “signalling aptamer” ATP1 in buffer.

FIG. 2. ATP binding to ATP-selective “signalling aptamer” ATP2 in buffer.

FIG. 3. ATP binding to ATP-selective “signalling aptamer” ATP3 in buffer.

FIG. 4. Particle size distribution of aptamer nanobiosensor comprising ATP-selective “signalling aptamer” ATP1 incorporated within polyacrylamide nanoparticle.

FIG. 5. ATP binding to aptamer nanobiosensor comprising “signalling aptamer” ATP1.

FIG. 6. ATP binding to aptamer nanobiosensor comprising “signalling aptamer” ATP2.

FIG. 7. Comparative nuclease stability of free “signalling aptamer” and nanobiosensor.

FIG. 8. Leaching of “signalling aptamer” from nanobiosensor.

FIG. 9. Phase contrast microscopy of yeast cells with incorporated intracellular aptamer nanobiosensor.

FIGS. 10A and B. Fluorescence microscopy of yeast cells in which incorporated intracellular aptamer nanobiosensors are “signalling”.

FIGS. 11A and B. Localization of intracellular aptamer nanobiosensor in yeast cytoplasm.

FIG. 12. Detection of intracellular ATP levels in yeast cells in vivo using an incorporated aptamer nanobiosensor.

FIG. 13. ATP nanobiosensor melting curve.

FIG. 14. ATP nanobiosensor calibration curve.

FIGS. 15A and B. Fluorescence microscopy and spectroscopic analysis of yeast cells bearing nanobiosensors functionalized with cell penetrating peptide.

FIG. 16A). The sequence of the aptamer switch probe used in this study and a schematic representation of signaling.

FIG. 16B). Probe signal upon addition of indicated final concentrations of ATP in selection buffer (20 mM phosphate buffer, pH=7.4; 140 mM KCl: 10 mM NaCl; 5 mM MgCl2, and 5.5% (w/v) glucose).

FIG. 16C) Nucleotide selectivity of the aptamer switch probe has been investigated by a titration in selection buffer.

FIG. 17. Kinetic measurements of ATP consumption by Hexokinase. 17A) First 3 mM ATP and then 2 units of hexokinase were added into a solution of 50 nM ATP aptamer switch probe in assay buffer (50 mM K2PO4, pH=7.0, 5 mM MgCl2, 20 mM glucose) at the indicated times. 17B) The initial velocities of ATP consumption (v0) were plotted for each ATP starting concentration.

FIG. 18A). Aptamer switch probe was embedded in 30 nm nanoparticles for protection against nucleases.

FIG. 18B). The ATP consumption by yeast cell extract was monitored by aptamer probe with or without nuclease inhibitor, and by aptamer probe embedded in nanoparticles in assay buffer with 3 mM ATP.

FIG. 18C). The initial rates of ATP consumption by cell extracts were plotted against ATP concentration.

### DETAILED DESCRIPTION OF THE INVENTION

As used herein the following terms have the following meanings:

“Average particle size” refers to the mean diameter of particle size distribution determined by correlation of dynamic light scattering data with assumption of approximately spherical particles.

“Signalling aptamer” refers to a ligand-selective aptamer sequence comprising fluorophoric, chromophoric or other means for providing spectroscopic signalling that is proportional to ligand binding.

Some embodiments provide an aptamer nanobiosensor comprising one or more aptamer sequences incorporated within a nanoparticle comprising polyacrylamide or other suitable polymer.

Other embodiments provide an aptamer nanobiosensor suitable for intracellular monitoring in vivo comprising one or more signalling aptamers incorporated within a nanoparticle comprising polyacrylamide or other suitable polymer. Preferred embodiments typically have average particle size less than about 50 nm.

Other embodiments provide biosensors suitable for use in cell-free systems and as affinity chromatography media.

Still other embodiments provide a nanobiosensor comprising a functional nucleic acid, such as a nucleic acid enzyme, or a combination of an aptamer and a nucleic acid enzyme, adapted for signalling and incorporated within a nanoparticle.

### Preparation of Signalling Aptamers.

Suitable signalling aptamers generally comprise 20-100 nucleotides.

Aptamers for target molecules of interest may be identified using “wild-type” or modified DNA or RNA libraries in a variety of SELEX and modified SELEX methods known in the art, including all of the methods described and/or cited in “The Aptamer Handbook: Functional Oligonucleotides and their Applications”, S. Klussman, editor, Wiley-VCH, 2006, which is hereby incorporated by reference in entirety.

Aptamers may also be identified using any of the specialized methods described in US20090004667, US20070243529, US20060121489, US20050142582, US20040086924, U.S. Pat. No. 7,329,742, U.S. Pat. No. 5,475,096, U.S. Pat. No. 5,270,163, U.S. Pat. No. 5,707,796, U.S. Pat. No. 5,580,737, U.S. Pat. No. 5,567,588, U.S. Pat. No. 5,496,938, U.S. Pat. No. 5,683,867 and WO91/19813, each of which is hereby incorporated by reference in entirety.

Alternatively, known aptamers including but not limited to any of those identified in Table 1 may be used.

TABLE 1
Suitable aptamer sequences.

| SEQ | | | |
|---|---|---|---|
| ID | | | |
| No. | Target | SEQ | Ref |
| 1 | Theophylline | GGCGAUACCAGCCGAAAGGCCCUUGGC | 14 |
| | | AGCGUC | |
| 2 | Cocaine | GACAAGGAAAATCCTTCAATGAAGTGG | 15 |
| | | GTC | |
| 3 | ATP | CACCTGGGGGAGTATTGCGGAGGAAGG | 16 |
| | | TT | |
| 4 | cAMP | GGAAGAGATGGCGACTAAAACGACTTG | 17 |
| | | TCG | |
| 5 | Citrulline | GACGAGAAGGAGUGCUGGUUCUACUAG | 18 |
| | | CGGUUAGGUCACUCGUC | |
| 6 | Arginine | GGGATCGAAACGTAGCGCCTTCGATCC | 19 |
| | | CGCATGACCAGGGCAAACGGTAGGTGA | |
| | | GTGGTCATGC | |
| 7 | Tobramycin | GGGUGACUUGGUUUAGGUAAUGAGUCA | 20 |
| | | CCCGGGACGAGGUUUAGCUACACUCGU | |
| | | CCC | |

In other embodiments, suitable aptamers may be identified by in silico methods and other methods.

DNA aptamers may be initially identified using DNA libraries or may be DNA equivalents of RNA aptamer sequences. RNA aptamers may be initially identified using RNA libraries or may be RNA equivalents of DNA aptamer sequences.

Identified aptamers may be further processed, for example by 5′ and 3′ end mapping to identify a “minimized” binding molecule. Aptamers may be further modified by addition of 3′ and/or 5′ capping structures such as inverted thymidine, biotin, alkylamines, polycations, proteins, fatty acids, PEG, cholesterol or other groups. Aptamers may be modified by other substitutions, such as 2′ modifications, base substitutions, phosphate substitutions, PEGylation, addition of specific nucleotide sequences at terminal ends or other modifications. As used herein the term “identified aptamer” relates to initial binding molecules characterized by SELEX methods as well as to minimized and/or further modified molecules.

Identified aptamers can be converted to signalling aptamers by a variety of different methods known in the art. In converting identified aptamers to signalling aptamers, some change in selectivity may be experienced. It will be readily understood that where one approach to signalling proves detrimental to specificity, an alternative approach may be pursued.

In one approach to signalling, aptamers can be converted to aptamer switch probes by the methods described in [21], which is hereby incorporated by reference in entirety. Briefly, the probe comprises a suitable aptamer, a short DNA or RNA sequence complementary to a short section of the aptamer, and a PEG linker connecting these two elements. In preferred embodiments, the hybridizing sequence is 6-10 nucleotides in length, while the PEG linker comprises 4-10 monomer residues. Alternatively, short complementary hybridizing sequences may be added at both 3′ and 5′ ends of the aptamer, with or without PEG linker. A fluorophore and a fluorescence quencher are covalently attached at the two termini of the conjugated nucleotide sequences.

Suitable fluorophores include at least any commercially available DYLIGHT™ (Thermo Scientific), ALEXA FLUOR™ (Molecular Probes), CY-DYE™ (GE Healthcare), LI-COR™ (LiCor Biosciences), CAL FLUOR™ (Biosearch Technologies), LC™ (Roche Applied Sciences), QUASAR™ (Biosearch Technologies), OYSTER™ (Integrated DNA Technologies), VIC™ (Applied Biosystems) and NED™ (Applied Biosystems) dyes and other fluorescent compounds known in the art or subsequently reported. Suitable quenches include at least any commercially available ECLIPSE™ (Epoch Biosciences), BHQ “Black Hole Quench”™ (Biosearch Technologies), DDQ “Deep Dark Quench”™ (Eurogentec), IOWA BLACK™ (DNA Technologies) or QSY™ (Molecular Probes) quench and other fluorescence-quenching compounds known in the art or subsequently reported.

In the absence of a target molecule, the short complementary sequence or sequences will hybridize, keeping the fluorophore and quench in close proximity. When the probe encounters its target, however, specific binding disturbs the intramolecular hybridization and moves the quench away from the fluorophore, restoring fluorescence and providing “signalling” of a binding event.

In alternative approaches, aptamers can be modified by addition of “molecular beacon” structures, such as those described in [21 and 22] which are hereby incorporated by reference in entirety. Typically, molecular beacons are formed by short, complementary extender sequences covalently attached at the 3′ and 5′ ends of a suitable recognition aptamer. In preferred embodiments, the extender sequences are 6-20 nucleotides in length.

The extender sequences may form a secondary structure such as a stem loop. A fluorescence/quench pair is attached at the 3′ and 5′ ends of the extender sequences. Target binding displaces the fluorescence/quench coupling and restores fluorescence, thereby providing “signalling” of a binding event.

In still other alternative approaches, aptamers can be modified by monochromophores, or other signalling moieties, such as those described in [23 and 24], which are hereby incorporated by reference in entirety. Typically a single fluorophore is covalently attached at a position in a suitable aptamer that experiences significant structural reorganization upon target binding. The fluorescence change associated with the target bound state is readily detectable, thereby providing “signalling” of a binding event.

In some embodiments, fluorophores, or fluorophore/quench pairs, or other “signalling” moieties, may be incorporated in the molecules that comprise the initial screening library from which an aptamer is selected. This can be advantageous, since no subsequent change in specificity is likely to be encountered.

In some embodiments, radiologic imaging modalities may be incorporated within the aptamer structure, either by modification of an identified aptamer or by a selection process that incorporates imaging modalities within the SELEX library. For example, for Single Photon Emmission Computer Tomography (SPECT), Tc-99, I-123 or In-111 may be covalently attached to an identified aptamer, or for positron emission tomography (PET), F-18, C-11 or Ga-68 may be used.

In still other embodiments, aptamers can be modified by covalent attachment of “signalling domain”, such as the malachite green RNA aptamer which can be joined to the “recognition domain” aptamer as described by US20060172320, which is hereby incorporated by reference in entirety. Binding of the target ligand to the “recognition domain” alters fluorescence properties of the bound fluorophore, thereby providing “signalling” of a binding event.

Many other strategies for obtaining fluorescence or other “signalling” of an aptamer binding event can readily be imagined by one skilled in the art.

### Preparation of Other Functional Nucleic Acids Adapted for Signalling.

In addition to aptamers, other functional nucleic acids such as nucleic acid enzymes and combinations of nucleic acid enzymes and aptamers may be adapted for signalling. DNAzymes and ribozymes can be selected using a variety of methods known in the art. For review see [24], which is incorporated by reference.

Nucleic acid enzymes can be utilized as sensors, typically where the target molecule is a co-factor or an inhibitor of the catalytic reaction.

Any of the variety of schemes known in the art for adapting nucleic acid enzymes for signalling may be used. Typically, various combinations of fluorophore/quench pairs are incorporated within the nucleic acid structure. Target binding and/or catalysis alter quench characteristics and give rise to concentration dependent signal. For review see [24].

### Incorporation of Signalling Aptamers in Nanoparticles.

Surprisingly, both DNA and RNA signalling aptamers survive the rigorous polyacrylamide encapsulation reaction with essentially full functionality. Suitable signalling aptamers can be incorporated within a polyacrylamide nanoparticle using a microemulsion polymerization method adapted from the methods described in [25, 26 and 27] which are hereby incorporated by reference in entirety.

In some embodiments, a reference dye embedded in the nanoparticle along with a suitable signalling aptamer provides a constant spectroscopic signal against which the target-binding signal may be normalized. Any suitable reference dye may be used, including, for example, 2′,T-Difluorofluorescein, Texas Red, and sulphorhodamine 101.

In preferred embodiments, signalling from aptamer switch probes or from aptamers derivatized with molecular beacon structures can be detected by time-resolved fluorescence. Fluorescence lifetime measurements in this context provide self-normalizing signals.

In preferred embodiments, the microemulsion polymerization process is conducted by (i) preparing an aqueous phase of the microemulsion by adding an aqueous solution comprising materials to be embedded in nanoparticles to an aqueous solution comprising both acrylamide monomer and an appropriate cross-linking agent, such as N,N′-methylene-bisacrylamide, (ii) preparing an oil phase containing a hydrocarbon liquid and an appropriate surfactant or surfactant mixture to form an inverse microemulsion consisting of small aqueous monomer droplets dispersed in the continuous oil phase and (iii) subjecting the acrylamide monomer microemulsion to polymerization. Optionally, one or more additives may be included in the aqueous phase to extend storage stability or impart other properties to the nanoparticles. Such additives may include, for example, metal chelating agents, glycerol, urea, antimicrobial agents, sodium dodecyl sulfate or other agents.

The surfactant-coated, nanometer sized reverse micelles formed in the microemulsion act as nanoreactors for polymerization of acrylamide monomers and, also provide a steric barrier that inhibits polymerization between micelles. Acrylamide monomer, cross-linking agent, and materials to be embedded, such as a suitable signalling aptamer and, optionally, one or more reference dyes, are incorporated fully within the reverse micelles. The polymerisation reaction and formation of nanoparticles occurs in the aqueous core of the micelles. The final size of polymerized nanoparticles is approximately the size of this aqueous core.

The size of the reverse micelles and, thus, the final size of the polyacrylamide nanoparticles is primarily determined by the volume ratio of surfactant to aqueous phase. In general, a higher volume ratio of surfactant to aqueous phase results in formation of smaller micelles and, thus, in smaller polymerized nanoparticles. The use of two types of surfactants, for example, AOT (sodium bis-2-ethylhexylsulphosuccinate), and Brij30 (polyoxyethylene 4 lauryl ether), also helps keep initial monomer micelle sizes very small.

Typical surfactants useful in the practice of this invention may be anionic, cationic or nonionic. Preferred surfactants include sodium dioctyl sulfosuccinate, polyoxyethylene-4-lauryl ether, sorbitan monooleate, polyoxyethylene, sorbitan monooleate, sodium dioctyl-sulfosuccinate, sodium bis-2-ethylhexylsulphosuccinate, oleamidopropyldimethyl amine, sodium isostearyl-2-lactate and other surfactants.

Any suitable organic solvent may be used to form the organic phase, preferably hexane.

In some embodiments, other suitable homopolymers or copolymers known in the art may be used for encapsulation, including at least poly(ethylene-vinyl acetate) copolymer, crosslinked polystyrene, poly(pyrrole), poly(L-lysine), and poly(ethylene glycol diacrylate).

In some embodiments, it may be advantageous to provide biodegradable biosensors using biodegradable homopolymers or copolymers to encapsulate including polyesters, such as poly(vinyl alcohol) (PVA) and polymers derived from lactic acid enantiomers, glycolic acid, and ε-caprolactone, including poly(lactic acid) (PLA), poly(glycolic acid) (PGA), and poly(lactic and glycolic copolymer) (PLGA); poly(ortho)esters including poly(ortho)esters I, II and III; poly(ether esters) such as polymers derived from poly(ethylene glycol), terephthalate, butylene succinate and butylene terephthalate including poly(ethylene glycol) terephthalate (PEGT)/poly(butylene terephthalate) (PBT) copolymers; poly(alkyl-2-cyanoacrylates), polyamides, poly(ethylene oxide) (PEO), and polyanhydrides.

In embodiments that utilize polyacrylamide as embedding material, pore size of the polyacrylamide matrix is primarily determined by acrylamide concentration and, to a lesser extent, N,N′-methylenebisacrylamide concentration in the microemulsion aqueous phase. Pore size can be minimized by keeping acrylamide concentration close to limits of aqueous solubility of acrylamide monomers. Smaller pore sizes prevent embedded aptamers and dyes from leaching out of the nanoparticle matrix. Monomer concentration in the microemulsion aqueous phase can affect final average particle size. If monomer concentration is decreased, average particle size can also be decreased. The concentration of dyes and aptamers in the microemulsion aqueous phase can affect monomer solubility and, thus, average particle size of the final polymerized particles. The presence of large amounts of dyes and/or aptamers impedes polymerization. Furthermore, the concentration of components for incorporation can alter solubility of acrylamide monomers and thereby affect average particle size of the polymerized particles. The amounts of signalling aptamer and, optionally, reference dye used can affect target-binding detection limits.

The particle size distribution and average particle size of the polymerized nanoparticles can be determined by dynamic light scattering, for example, as described by any of references [28, 29 and 30]. In preferred embodiments, nanoparticles may be sonicated and/or filtered to provide comparatively homogeneous particle size distribution.

In preferred embodiments, average particle size of the nanoparticles is less than 50 nm diameter. In other embodiments, average particle size may be less than 150 nm, or less than 100 nm.

In other embodiments, targeting ligands useful in targeting the nanoparticle and its contents to particular cells may be directly incorporated in the polymer matrix. For example, acrylamide monomers or mini-polymers can be conjugated to polypeptide targeting sequences by any method known in the art, including, but not limited to, the methods disclosed by [31, 32, 33, and 34]. Acrylamide monomers or mini-polymers derivatised with targeting sequences and/or other targeting agents can be added to the aqueous phase of the microemulsion prior to polymerization. Alternatively, targeting sequences and/or other targeting agents may be added to the nanoparticles after polymerization. For example, the nanoparticles may be coated with glycosylated and/or unglycosylated polypeptide sequences as described by references [35 and 36].

### Incorporation of Other Functional Nucleic Acids Adapted for Signalling in Nanoparticles.

Methods for incorporation of aptamers within nanoparticles can also be applied to other functional nucleic acids adapted for signalling.

Incorporation of Nucleic Acid Nanobiosensors within Cells.

Nanobiosensors of the present invention may be used in cell culture in vivo. Nanobiosensors may be incorporated into cells by any method known in the art including but not limited to balistic insertion (gene gun), microinjection, electroporation, targeting sequence delivery (e.g., conjugation with cellular uptake peptides, and targeted liposomal delivery, for example, as described in [37, 38, 39, and 40].

Suitable cells include at least yeast, bacteria, and cultured mammalian cells, such as human cells, including at least keratinizing epithelial cells, barrier epithelial cells, exocrine secretory epithelial cells, hormone secreting cells, metabolizing and storage cells, barrier function cells, epithelial cells lining closed internal body cavities, ciliate cells, extracellular matrix secretion cells, contractile cells, sensory cells, autonomic neuron cells, peripheral neuron cells, lens cells, pigment cells, nurse cells, and blood and immune cells such as erythrocytes, megakaryocytes, monocytes, macrophages, osteoclasts, dendritic cells, microglial cells, granulocytres, mast cells, helper T cells, suppressor T cells, cytotoxic T cells, natural killer T cells, B cells, natural killer cells, reticulocytes and stem cells.

Nanobiosensors of the present invention can be used in biopsy samples, organ slices, isolated perfused organs, organotypic cultures, organs in situ, whole animals in vivo and in other circumstances in which aptamer-target binding measurements are desired in only one or some cell types present in a mixed cell system. For example, the nanosensors can be targeted for intracellular incorporation to particular normal or cancerous cells by incorporation of targeting sequences in the nanoparticles, as described by the following references, each of which is hereby incorporated by reference herein in entirety: US published application 2005/0042298, US published application 2006/0018826; US published application 2006/0034925.

In one embodiment, the invention provides a cell comprising an embodiment of the nanobiosensor as described herein.

### Use of Nanobiosensors in Cell-Free Systems.

In some embodiments, it may be advantageous to use nanobiosensors according to the invention in a cell-free system, for example to provide accurate measurements in blood or other liquid samples or as an affinity chromatography medium.

The nanobiosensors can be arranged into multiple-well arrays or similar multi-sample detection systems suitable for high-throughput analysis. The nanobiosensors can be used in a variety of different sample fluids, including at least blood, plasma, urine, cerebrospinal fluid, tissue extracts, cell extracts and other body fluids as well as in environmental samples such as water, soil, and plant extracts, and production samples, such as bio-fermentation broths. Target ligands of interest include at least metabolites, environmental pollutants, drugs, drug metabolites and other xenobiotic compounds.

As affinity chromatography media, the nanobiosensors provide convenient methods for purification of fine chemicals, natural compounds, products of fermentation broths, and other target ligands of interest.

### Detection and Quantification of Target Binding Using Intracellular Aptamer Nanbiosensor.

In some embodiments, intracellular ligand measurements can be determined using calibration curves with normalized fluorescence signals, where the nanobiosensor comprises a reference dye, or where fluorescence is detected with time-resolution.

In other embodiments, intracellular ligand measurements can be determined using indirect normalization. Fluorescent nanobiosensors will exhibit increasing fluorescence with increasing temperature from 25° C. up to 50° C., as the hairpin or other structure of the aptamer probe “melts”. Ligand-bound sensors typically exhibit a stabilized “melting curve” with enhanced fluorescence relative to unbound sensor up to a particular temperature. This temperature can be readily determined. Fluorescence measurements at this temperature provide an estimate of “minimum fluorescence” for the particular cells used. A calibration curve can be readily determined in buffer relating F/F0 (fluorescence/“minimum fluorescence”) for varying concentrations of ligand. Intracellular measurements of fluorescence at normal temperature and at “minimum fluorescence” temperature provide an estimate of F/F0 which can be related to calibration curves to provide an estimate of intracellular ligand concentration.

A variety of intracellular target ligands are suitable for detection and quantification using the nanobiosensors, including at least any of the metabolites identified in the Human Metabolome database as well as xenobiotic compounds. The nanobiosensors can be used, for example, in drug development studies to quantify small molecule concentration in specific tissues from experimental animals or in biopsies or other samples from humans.

### EXAMPLES

### 1. Preparation of Signalling Aptamers

### ATP1

Texas RedX-CACCTGGGGGAGTATTGCGGAGGAAGGTT-
(CH2CH2O)₃₆-CCAGGTG-BHQ2

An ATP signalling aptamer probe (ATP1) was designed using SEQ ID No. 3, which is an aptamer region selected previously by Huizenga [16]. A similar switch probe incorporating this sequence was described previously by Tang et. al. [21]. The aptamer SEQ ID No. 3 was modified at the 3′ end with a polyethylene glycol linker (6-mer) and a 7-mer hybridizing sequence which is complementary to the first 7 nucleotides of the aptamer sequence, and with the fluorescence quench BLACK HOLE 2™ (Biosearch Technologies). At the 5′ end, the aptamer SEQ ID No. 3 was modified by the fluorophore “Texas Red” (sulforhodamine 101 acid chloride).

### ATP2

Cy3-GGAAGAGATGGCGACTAAAACGACTTGTCG-(CH2CH2O)₃₆-
CTCTTCC-IowaBlackRQ

An ATP signalling aptamer probe (ATP2) was designed using SEQ ID No. 4. This is a DNA sequence corresponding to the cAMP-selective RNA aptamer sequence reported by Koizumi [20]. SEQ ID No. 4 was modified at the 3′ end with a polyethylene glycol linker (6-mer), a 7-mer hybridizing sequence, and the fluorescence quench IOWA BLACK FQ™ (Integrated DNA Technologies). At the 5′ end, the aptamer SEQ ID No. 10 was modified with the fluorophore Cyanine 3 (Cy3).

### ATP3

AlexaFlour488-ACGAGGGGACGAAAGTCCCCGGACAATCAGACACG
GTC-(CH2CH2O)₃₆-CCCTCGT-BHQ2

An ATP signalling aptamer probe (ATP3) was designed using SEQ ID No. 8. This is a DNA sequence corresponding to the ADP-selective RNA aptamer sequence reported by Srinivasan et al. [41]. SEQ ID No. 8 was modified at the 3′ end with a polyethylene glycol linker (6-mer), a 7-mer hybridizing sequence, and the fluorescence quench BLACK HOLE 2™ (Biosearch Technologies) fluorescence quench. At the 5′ end, the aptamer SEQ ID No. 12 was modified with the fluorophore ALEXA FLUOR 488™ (Molecular Probes).

### 2. Calibration of ATP-Sensitive DNA Signalling Aptamer in Buffer

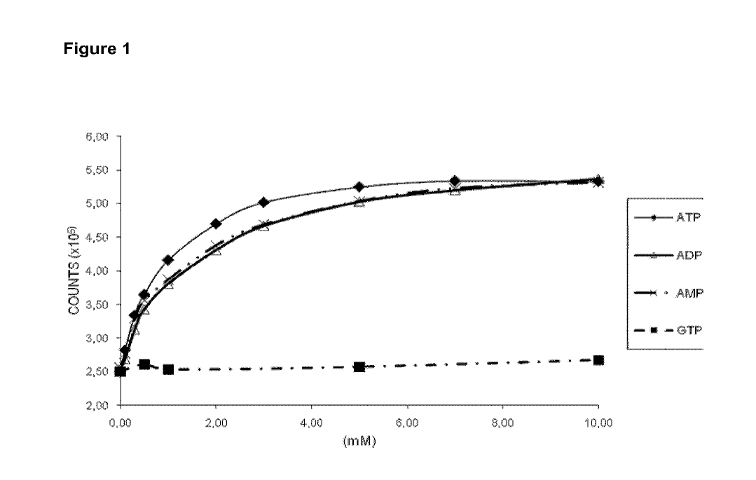

The fluorescence counts of ATP1 signalling aptamer solution was recorded as a range of target solution added by continuous mixing in a fluorescence spectrometer (Edinburgh Instruments). The aptamer solution was 25 nM probe in 100 mM KPO₄ buffer and 1 mM MgCl₂. The excitation wavelength was 590 nm and emission measured at 610 nm. The response of aptamer probe was selective for ATP, ADP and AMP over GTP as reported in the original publication [41]. The response of probe for ATP, ADP and AMP was at similar levels. Note that AMP, ADP and ATP are different by addition of one phosphate group sequentially. Equilibrium constants (KD) were determined as 0.92 mM for ATP and 1 mM for both ADP and AMP. FIG. 1 shows results for signalling aptamer ATP1 titrated using 25 nM ATP1 aptamer in 100 mM KPO4 buffer and 1 mM MgCl2 with ATP, ADP, AMP, and GTP. Emission at 610 nm was recorded after excitation at 590 nm.

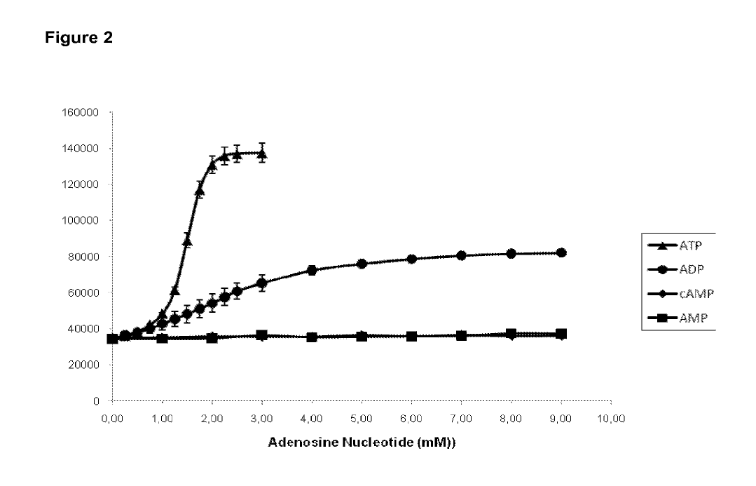

The fluorescence counts of ATP2 signalling aptamer solution was recorded as a range of target solution added by continuous mixing in a fluorescence spectrometer (Edinburgh Instruments). The aptamer solution was 25 nM probe in 10 mM K₂HPO₄—KH₂PO₄ buffer and 1 mM MgCl₂. The excitation wavelength was 545 nm and emission measured at 565 nm. The response of aptamer probe was selective for ATP over ADP, AMP and cAMP. Equilibrium constants (KD) were determined as 1.45 mM for ATP and 2.50 mM for ADP. There was no response for AMP and cAMP titration. FIG. 2 shows results for signalling aptamer ATP2 titrated using aptamer in 10 mM K₂HPO₄—KH₂Pa₄ buffer and 1 mM MgCl₂ with ATP, ADP, AMP, and cyclicAMP (cAMP).

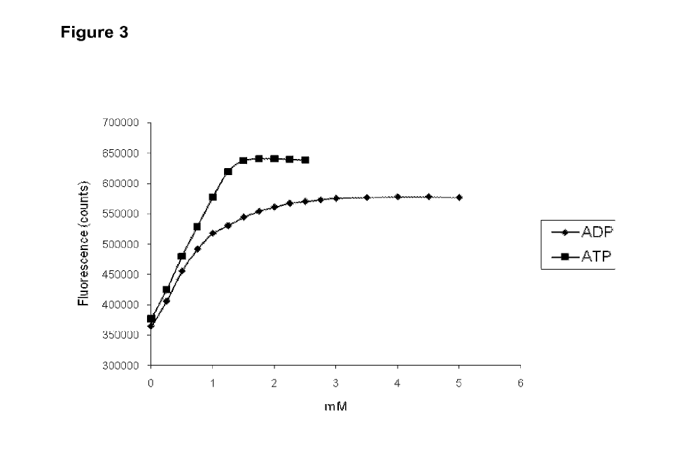

The fluorescence counts of ATP3 signalling aptamer solution was recorded as a range of target solution added by continuous mixing in a fluorescence spectrometer (Edinburgh Instruments). The aptamer solution was 25 nM probe in 10 mM K₂HPO₄—KH₂PO₄ buffer and 1 mM MgCl₂. The excitation wavelength was 480 nm and emission measured at 520 nm. Results for ATP3 are shown in FIG. 3. The response of aptamer probe was similarly selective for both ATP and ADP.

### 3. Incorporation of ATP-Sensitive DNA Signalling Aptamer into Polyacrylamide Nanoparticles

The nanosensors were prepared by inverse microemulsion polymerization. 3.08 g of AOT and 1.59 g of Brij 30 was dissolved in 45 mL of hexane. This solution was sonicated for 1 hour with a gentle argon flow to remove oxygen. 1.35 g of acrylamide and 0.4 g of N,N-methylenebisacrylamide was dissolved in 4.5 mL of 10 mM sodium phosphate buffer pH=7.25 with sonication. 280 mL of aptamer was mixed with 2.0 mL of acrylamide solution, and 2.0 mL of this solution was added to the hexane solution and stirred for 15 min. under an argon atmosphere allowing the microemulsion to form. The polymerization was initiated by the addition of 50 mL of 10% w/v sodium bisulphite in 10 mM sodium phosphate buffer pH=7.25 and left to proceed under an argon atmosphere for 3 h. The hexane was then removed in vacuo and the nanosensors were precipitated by the addition of 100 mL of ethanol. The suspension was transfered to an Amicon ultrafiltration cell Model 2800, filtered and washed with 4×100 mL of ethanol. The particles were resuspended in 50 mL of ethanol which was removed by filtration (0.025 mm nitrocellulose filter membrane) and the particles were dried in vacuo and stored at −18° C.

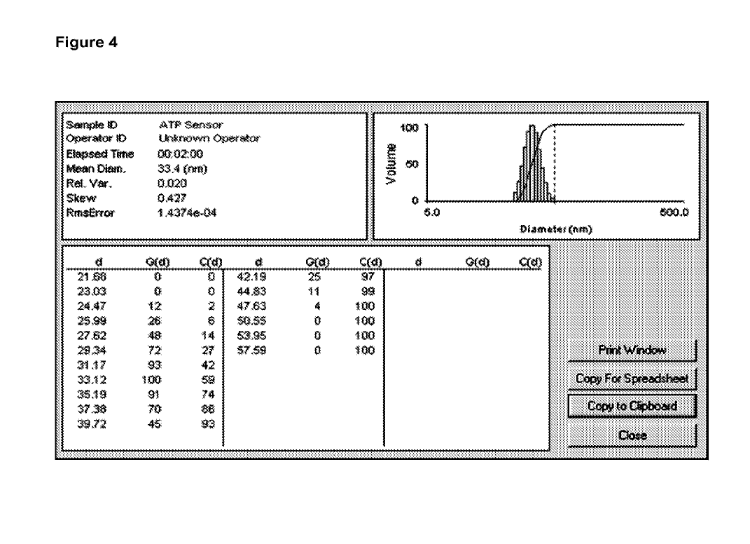

The particle size distribution of signalling aptamer ATP1 embedded in acrylamide nanoparticles was determined by dynamic light scattering in a BI-2000 (Brookhaven Instruments). The sample was 1.5 ml (1 mg/ml solution of particles) in water. Samples were sonicated and filtered through a 0.45 μm filters before measurements. Average particle size of nanoparticles containing aptamers was determined as 33.4 nm by dynamic light scattering. The average NNLS particle size distribution was determined by correlating scattering data. The diameter distribution of particles is shown in FIG. 4.

### 4. Calibration of Naniobiosensor Comprising ATP-Sensitive DNA Signalling Aptamer in Polyacrylamide Nanoparticle

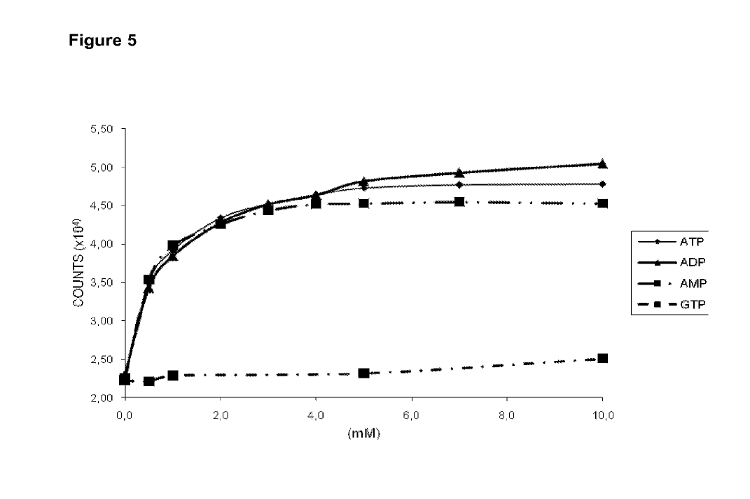

The responses of aptamer probes embedded in acrylamide nanoparticles were determined in similar experiments as for titration of probes in solution. For comparisons, 100 μg of nanoparticles containing ATP signalling aptamer sensor were solubilized in 100 mM KPO₄ buffer and 1 mM MgCl₂. The excitation wavelength was 590 nm and emission measured at 610 nm. The response of biosensor ATP1 is shown in FIG. 5. Titration of 100 μg of nanoparticles containing ATP aptamer sensor was conducted using the same conditions as described for FIG. 1. The response was selective for ATP, ADP and AMP over GTP similar to the results observed with probe in solution.

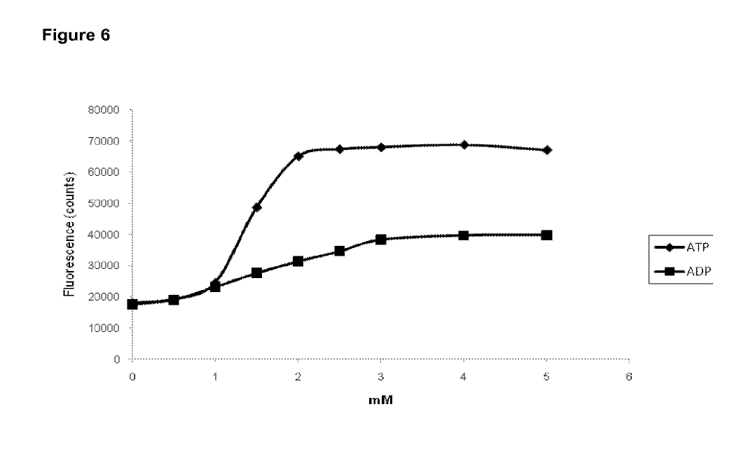

The fluorescence response of ATP2 biosensor particles in solution was recorded after a range of target solution was added by continuous mixing in a fluorescent spectrometer (Edinburgh Instruments). The aptamer nanosensor solution was 100 μg nanoparticles in 10 mM KCO₄ buffer and 1 mM MgCl₂. The excitation wavelength was 545 nm and emission measured at 565 nm. The response of biosensor ATP2 is shown in FIG. 6. Titration of ATP2 nanosensor was conducted in 10 mM K₂HPO₄—KH₂PO₄ buffer and 1 mM MgCl₂ with ATP and ADP. The response was selective for ATP over ADP similar to aptamer response in solution. Equilibrium constants (KD) were determined as 1.5 mM for ATP and 2.10 mM for ADP.

### 5. Comparative Nuclease Stability of Free Signalling Aptamer and Nanobiosensor

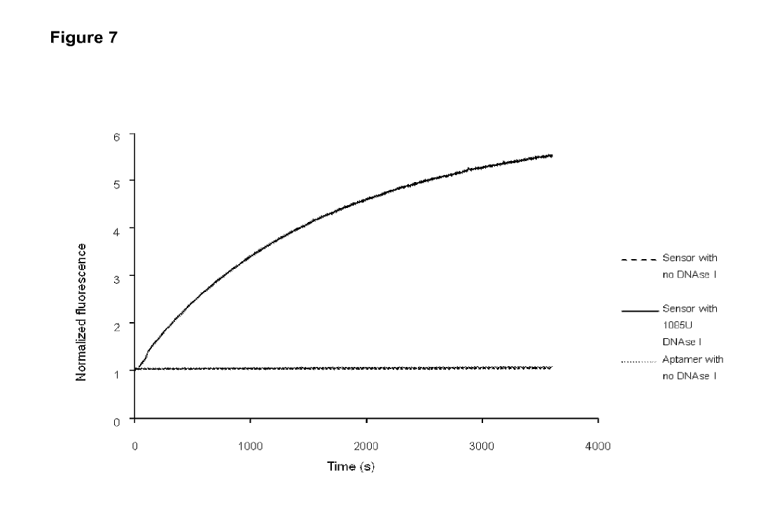

A 1 μM solution of ATP1 signalling aptamer in 10 mM sodium phosphate buffer with 1 mM MgCl₂ pH=7.25 and a 1 mg/mL solution of the ATP1 nanosensor in 10 mM sodium phosphate buffer with 1 mM MgCl₂ pH=7.25 was subjected to degradation with 1085 U of DNAse 1 for 1 h, with continuous fluorescence monitoring. Both the free aptamer and the aptamer nanosensor solutions were monitored under DNAse free conditions as a control. Results are shown in FIG. 7. Shown are fluorescence increase as a function of time with and without DNAse I for both the free and the encapsulated signalling aptamer. As shown, complete degradation of free signalling aptamer results in a 17-fold increase in fluorescence, whereas the increase in fluorescence with encapsulated signalling aptamer is only 5.5-fold. This shows that encapsulation in polyacrylamide nanoparticles protects signalling aptamers from nuclease degradation. The partial degradation observed is likely due to degradation of aptamers placed close to, or partially sticking out of, the surface of the nanoparticle.

### 6. Leaching of Signalling Aptamer from Nanobiosensor

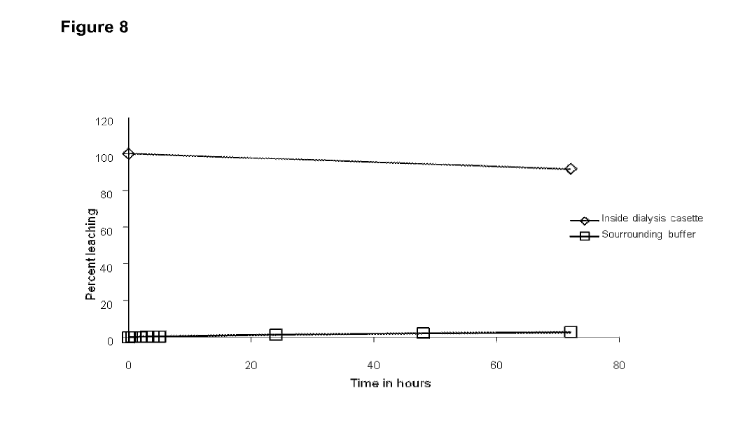

Leaching experiments were performed using a Slide-A-Lyzer 20K MWCO™ dialysis cassette (Thermo Fischer Scientific Inc., Rockford, USA) washed to manufacturers recommendations with 10 mM sodiumphosphate buffer pH=7.25. 1.0 mL of a 50 mg/mL solution of ATP1 nanosensors in 10 mM sodium phosphate buffer was injected into the dialysis cassette, and the cassette placed in 200 mL of sodium phosphate buffer with stirring. The initial fluorescence of both nanosensor solution and surrounding buffer were measured. Fluorescence of the surrounding buffer was then measured at times 0.5 h, 1 h, 2 h, 3 h, 4 h, 5 h, 24 h, 48 h and 72 h to evaluate the leaching ratio. As a control fluorescence of the solution inside the cassette was measured at 72 h. Results are shown in FIG. 8. As shown, leaching of aptamers from within the polyacrylamide nanoparticle is minimal. Only −5% of the aptamer leached out over 72 h.

### 7. Incorporation of Nanobiosensor into Yeast Cells In Vivo

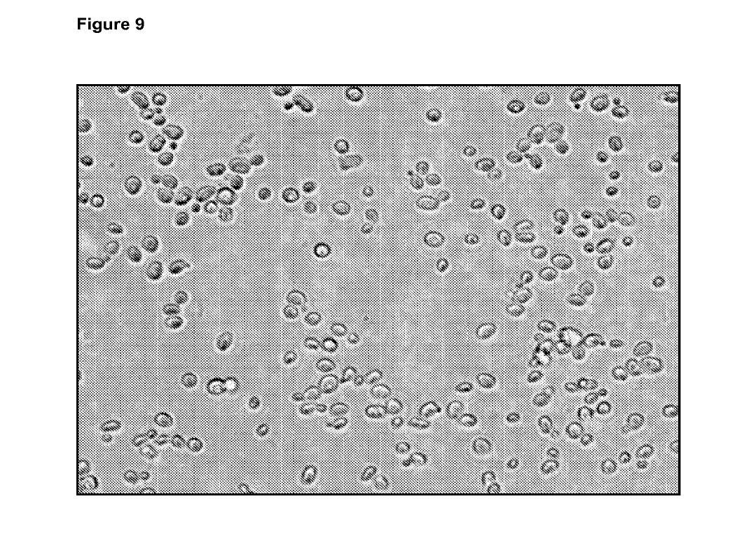

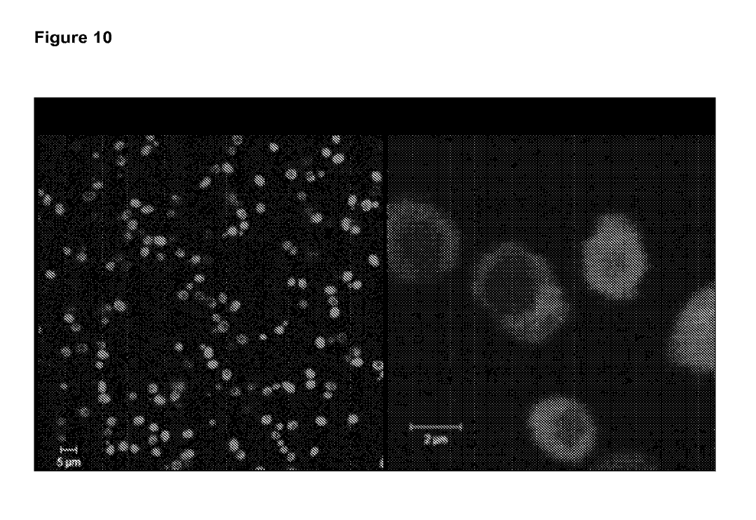

ATP1 signalling aptamers embedded in acrylamide nanoparticles were electroporated into yeast cells and examined by a Zeiss LSM 510 confocal microscope. Phase contrast microscopy images of the cells are shown in FIG. 9. The darkening within cells indicates that nanosensors signal presence of adenosine nucleotides (ATP, ADP and AMP). Fluorescence microscopy images of the cells at two different scales is shown in FIGS. 10A and 10B. The lighter contrast shows Texas Red fluorescence. The magnified picture in FIG. 10B indicates that aptamer nanosensors are distributed evenly within cytoplasm of cells.

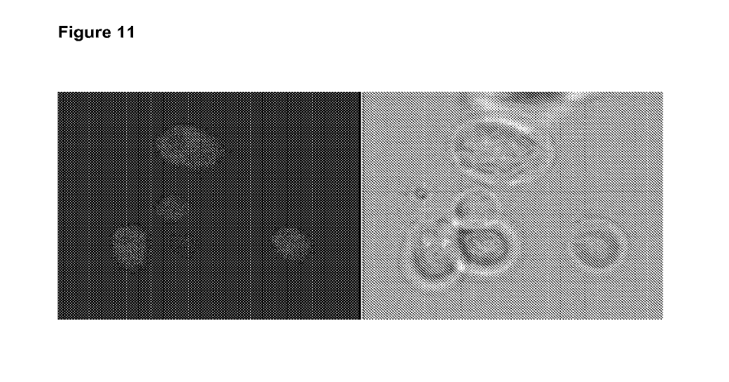

ATP3 signalling aptamers embedded in acrylamide nanoparticles were electroporated into yeast cells and examined by a Zeiss LSM 510 confocal microscope. Fluorescence microscopy images of the cells are shown in FIGS. 11A and 11B. FIG. 11B shows the brightfield microscopic image of the cells and FIG. 11A shows the fluorescence overlap. The lighter contrast in FIG. 11A shows Alexa Flour 488 fluorescence. The nanosensors in yeast cells signal presence of adenosine nucleotides (ATP and ADP).

### 8. Measurement of Intracellular ATP Level Using Aptamer Nanobiosensor in Yeast Cells In Vivo

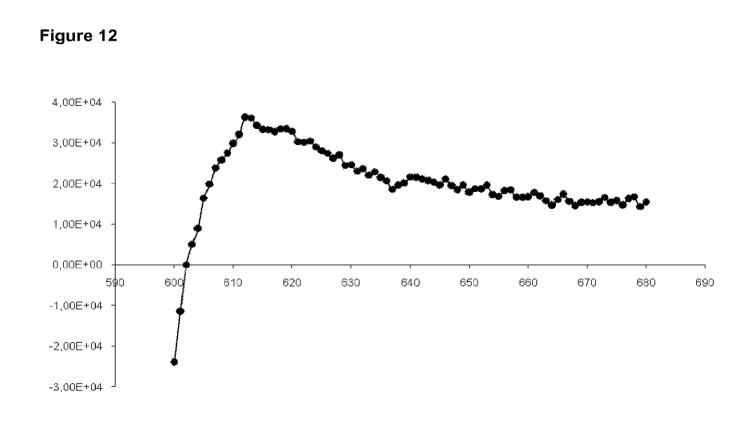

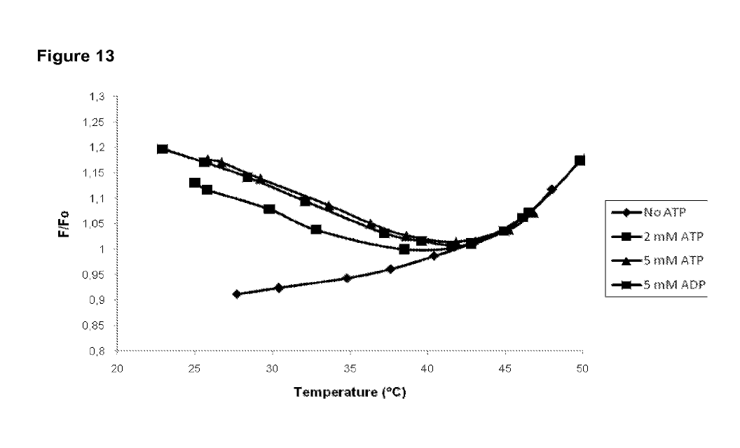

Emission spectrum of yeast electroporated with ATP1 nanosensors is shown in FIG. 12. The figure shows the difference spectra of Texas Red emission of electroporated cell over control cells. Nanosensor signals correspond to adenosine nucleotide concentration inside cells. The amount of total Adenosine nucleotides (ATP, ADP and AMP) in yeast was calculated as approximately 3.0 mM from fluorescence intensity by reference to calibration curves. A temperature melting curve analysis of sensor is shown in FIG. 13. As shown, increasing temperature from 25° C. up to 50° C. causes the hairpin structure of the aptamer probe to open resulting in increase in Texas Red fluorescence. With ATP-bound sensors, the hairpin structure is stabilized by release of ATP up to 43° C. Over 43° C., the aptamer probe hairpin starts to melt independent of ATP state of probe (ATP bound or free). Thus, the ATP binding site of probe is less stable than the hairpin region under 43° C.

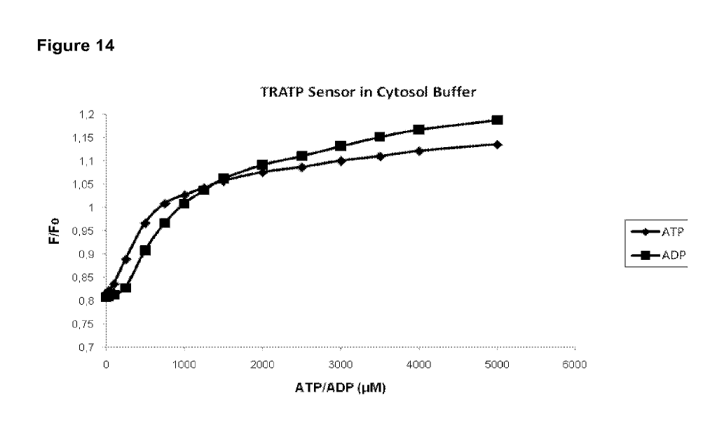

Fluorescence at 43° C. was used as the minimum fluorescence reference in calculations of ATP concentrations. A standard curve showing (F/Fo) vs target concentrations shown in FIG. 14 was used to calculate the concentration of nucleotides inside cells. For yeast experiments the values of F (25° C.) were 36300. F0 (43° C.) was 32110, corresponding to F/F0 of 1.1305, or about 3 mM by reference to the calibration curve shown in FIG. 14.

The amount of total nanoparticles in the cells shown in FIG. 12 can be approximated as about 5 nM aptamer, based on fluorescence intensity. Each nanoparticle is estimated to contain two aptamer probes on average based on the diameter of particles determined in Example 3. The number of cells used in electroporation experiments was counted as 1,125 billion in 2 ml samples as used in experiments. The average number of nanosensors in yeast cells was thus approximately 2700 per cell.

### 9. Synthesis and Intracellular Incorporation of Nanosensors Functionalized with Cell Penetrating Peptide

Nanosensors were prepared by inverse microemulsion polymerization. 3.08 g of AOT and 1.59 g of Brij 30 was dissolved in 45 mL of hexane. This solution was sonicated for 1 hour with a gentle argon flow to remove oxygen. 530 mg of acrylamide, 160 mg of N,N-methylenebisacrylamide and 20 mg of N-(3-aminopropyl) methacrylamide hydrochloride was dissolved in 2.0 mL of 10 mM sodium phosphate buffer pH=7.25 with sonication. 25 mL of Texas Red-dextran and 50 mL of BCECF-dextran was mixed with 2.0 mL of acrylamide solution, and 2.0 mL of this solution was added to the hexane solution and stirred for 15 min under an argon atmosphere allowing the microemulsion to form. The polymerization was initiated by the addition of 50 mL of 10% w/v sodium bisulphite in 10 mM sodium phosphate buffer pH=7.25 and left to proceed under an argon atmosphere for 3 h. The hexane was then removed in vacuo and the nanosensors were precipitated by the addition of 100 mL of ethanol. The suspension was transferred to an Amicon ultrafiltration cell Model 2800, filtered and washed with 4×100 mL of ethanol. The particles were resuspended in 50 mL of ethanol which was removed by filtration (0.025 mm nitrocellulose filter membrane) and the particles were dried in vacuo and stored at −18° C. For attachment of the peptides the bifunctional linker GMBS was used. 100 mg of nanosensors added to 5 mL of a 2 mM solution of GMBS in ethanol and stirred for 1 h. The nanosensors were collected by centrifugation and washed with 20% v/v water in ethanol. The nanosensors were resuspended in 5 mL of 10 mM sodium phosphate buffer pH=7.25 to which was added 5 mg of cell penetrating peptide (MAP peptide described in [42], SEQ ID No. 9). This solution was stirred for 2 h at RT and overnight at 4° C. The nanosensors were once again collected by centrifugation, washed with 20% v/v water in ethanol and filtered (0.025 mm nitrocellulose filter membrane). The particles were dried in vacuo and stored at −18° C.

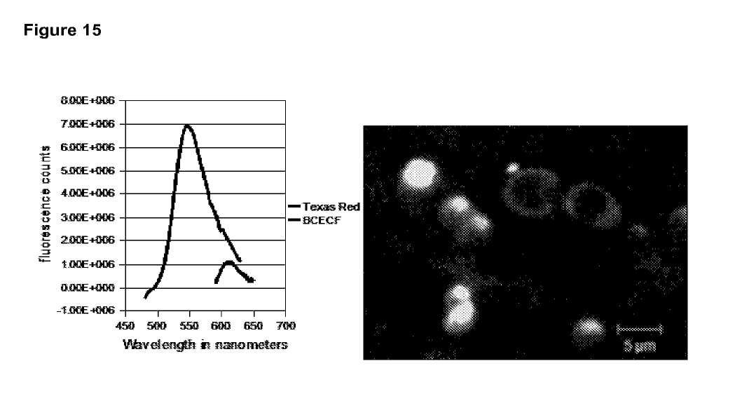

Yeast was incubated with the peptide-functionalized nanosensors for 1 h at 40 degrees. The cells were then washed and trypsinized and the intracellular incorporation of peptide-functionalized nanosensors confirmed by fluorescence spectroscopy and confocal microscopy, as shown in FIGS. 15A and 15B. FIG. 15A shows that yeast cells that have been incubated with the CPP functionalized nanosensors display a strong fluorescence emission at 540 nm and 610 nm consistent with uptake of the particles containing BCECF and Texas Red. FIG. 15B shows a confocal microscopy image of the same yeast cells at emission 540 nm corresponding to BCECF. This further provides evidence of the uptake of the CPP functionalized nanosensors.

### 10. Cell-Free Detection of Theophylline in Blood Samples

Theophylline (1,3-dimethylxanthine), a bronchial dilator and respiratory stimulator, is one of the most commonly used drugs for the treatment of symptoms of acute and chronic asthma conditions. It is one of the most frequently clinically monitored drugs in the USA because of its very narrow therapeutic range (20-100 mM). Serum or plasma theophylline concentrations have previously been determined using gas/liquid chromatography and immunoassays. However, these methods often overestimate concentration due to interference from structurally similar molecules such as caffeine and theobromine.

A theophylline-selective signalling aptamer can be prepared using SEQ ID No. 1 modified at the 3′ and 5′ ends as described in Example 1. The signalling aptamer can be incorporated in polyacrylamide nanoparticles as described in Example 3 and used to measure theophylline concentration in blood or serum samples, providing more precise measurements.

### 11. Nuclease-Resistant Affinity Chromatography

Moenomycin A is an antibiotic suitable for controlling Helicobacter pylori infections, which are the main cause of gastritis and ulcers in humans. Moenomycin A can be isolated from cultures of moenomicin-producing organisms using affinity chromatography matrix comprising nanobiosensors according to the invention. A moenomycin-selective aptamer can be prepared using SEQ ID No. 10. This is an RNA aptamer sequence reported in [43]. The aptamer can be incorporated in polyacrylamide nanoparticles as described in Example 3. Nanoparticles comprising aptamer encapsulated in polyacrylamide provides a suitable affinity chromatography medium. The target ligand can be eluted by change of pH.

All of the cited references are incorporated by reference herein.

The description of preferred embodiments and examples are representative, only, and not intended to limit the scope of the invention as defined by the claims herein.

### ATP4

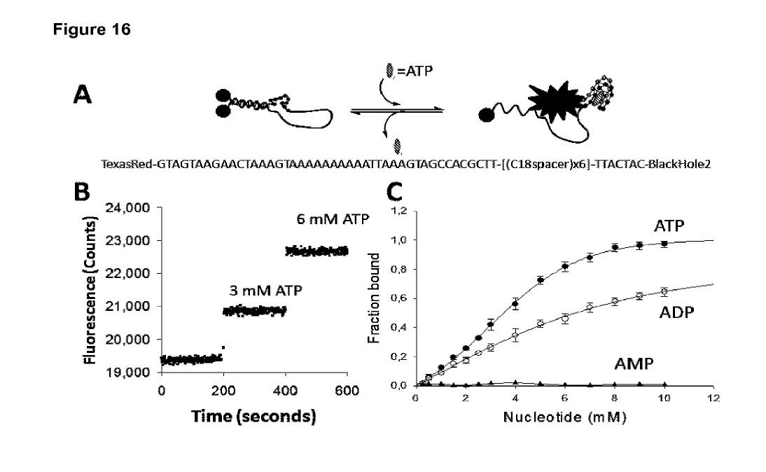

In the process of developing a nanobiosensor specific for ATP the inventors have selected a new ATP binding DNA aptamer sequence that preferentially binds ATP. The selected aptamer sequence was converted to a switch probe based on a design by Tang et. al. [53]. The probe is an intramolecular signal transduction aptamer design which consists of the aptamer sequence and a short hairpin forming a complementary sequence separated by a PEG linker. A fluorophore (Texas red) and quencher (Black Hole 2) are covalently attached at the two ends of the sequence. The interaction of ATP with the aptamer sequence disrupts the hairpin structure and leads to rearrangement of the structure separating the quencher and fluorophore from each other. This leads to an increase in fluorescence which depends on the concentration of ATP (FIG. 16A). The instant and reversible response can be obtained upon addition of ATP in solution containing the probe, indicating the usefulness of the new sensor in monitoring biological reactions involving ATP (FIG. 16B).

Another important criterion in selection was to identify aptamers with lower affinity considering that many enzymes catalyzing ATP consumption have KM values for ATP in the millimolar range and the level of intracellular ATP concentration is also in this range. Thus, the selection procedure used here was designed to seek a specific ATP aptamer with a dissociation constant in the micromolar range following a series of negative selections to eliminate ADP and AMP binding. One of the selected sequences was further characterized by Surface Plasmon Resonance analysis which indicated a Kd of 692 μM.

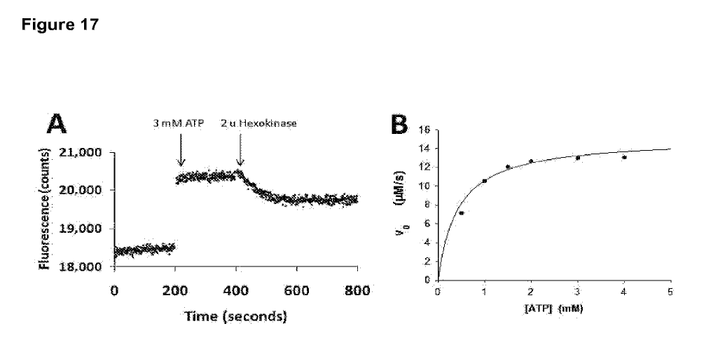

The switch probe prepared from the selected aptamer sequence was evaluated for its ability to discriminate between different adenine-nucleotides in vitro (FIG. 16C). The fluorescence change was larger for ATP binding compared to ADP binding in the target concentration range from 0.5 up to 8 mM. The weaker binding of ADP allowed monitoring of ATP changes. There was no significant response to AMP. Kd values were calculated as 3.22 mM for ATP and 4.37 mM for ADP. The results presented in FIG. 17C indicate that this new aptamer switch probe can be used to measure ATP concentrations in real-time. The inventors tested this postulate by following ATP concentration changes by establishing a kinase assay based on hexokinase activity. A single monotonic decrease has been observed after mixing ATP, sensor probe and hexokinase (FIG. 17A). The experiment in FIG. 17A was repeated using different activities of hexokinase or different initial concentrations of ATP and the observed initial rates were plotted.

The linear relationship between enzyme activity and reaction rates demonstrates that the probe is indeed able to monitor enzyme activities where ATP is a substrate. The Michaelis-Menten (KM) constant for ATP has been estimated as 0.47±0.11 mM by fitting a hyperbolic curve to the ATP titration plot (FIG. 17C). The calculated KM value was in good agreement with previous estimates of around 0.2 mM for hexokinase.

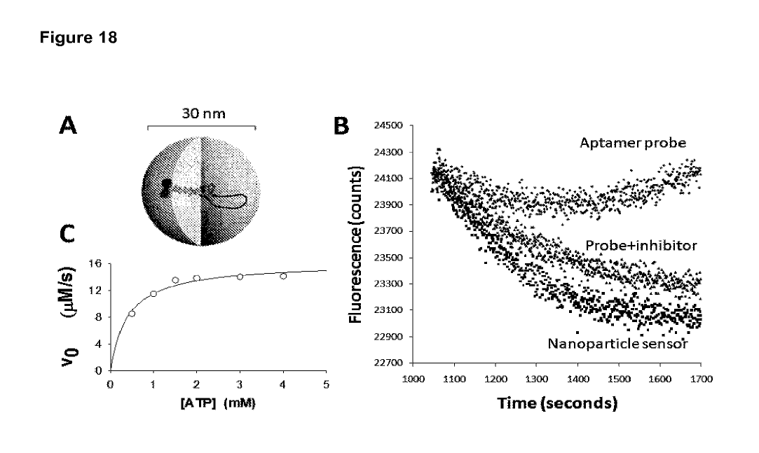

One of the major drawbacks of aptamer probes are their sensitivity to nucleases ubiquitously present in living environments. Although nuclease inhibitors can be used to overcome the degradation for in vitro applications, in vivo applications would need alternative solutions. Moreover, the use of nuclease inhibitors is not preferable because of the possibility of inhibiting the reaction of interest. To further enhance the usefulness of the new aptamer probe, an aptamer based nanobiosensor was prepared by encapsulating the ATP-specific aptamer in polyacrylamide nanoparticles (FIG. 18A). The porous and transparent nature of the polyacrylamide nanoparticles allows the rapid diffusion of small analytes like ATP in and out of the particles, but prevents the interaction of the embedded aptamer probe with nucleases in the environment. The nanoparticles with embedded aptamers were tested in vitro on the hexokinase system similar to the experiments with the naked aptamer probe (FIG. 17).

The enzyme's KM calculated from experiments with nanoparticle sensors was 0.40 mM and in good agreement with the constant calculated with the naked aptamer probe (FIG. 17C).

To further explore the utility of nanosensors in the context of realtime ATP monitoring, yeast cell extracts were prepared by passing cells through a French Press. The inventors observed that kinase assays with cell extract required addition of nuclease inhibitors along with aptamer probe to prevent degradation, but aptamer probes embedded in nanoparticles were able to measure kinase activity without inhibitors (FIG. 18B). KM of glucose-dependent kinases in the cell extract was determined as 0.69 mM for cell extracts (FIG. 18C).

The new aptamer selected in this study has been shown to be useful in monitoring kinetics of ATP involving reactions. The probe based on this aptamer can be used to monitor real-time changes from 0.5 to 8 mM, and hence extends the capabilities of most of the previously developed ATP sensors which have maximum limits of approximately 200 μM ATP. The aptamer probe was further improved to eliminate nuclease degradation by embedding it in polyacrylamide nanoparticles. The strategy used here for ATP monitoring can readily be applied to any metabolite of interest in cells.

### REFERENCES

1. De-los-Santos-Álvarez, N., Jesús Lobo-Castañón, M., Miranda-Ordieres, A. J., Tuñón-Blanco, P., “Aptamers as recognition elements for label-free analytical devices”, Trends in Analytical Chemistry, Vol. 27, No. 5, 2008.
2. Song, S., Wang, L., Li, J., Zhao, J., Fan, C., “Aptamer-based Biosensors”, Trends in Analytical Chemistry, Vol. 27, No. 2, 2008.
3. Mairal, T., Özalp, V. C., Sánchez, P. L., Mir, M., Katakis, I., O'Sullivan, C. K., “Aptamers: molecular tools for analytical applications”, Anal Bioanal Chem (2008) 390:989-1007.
4. Balamurugan, S., Obubuafo, A., Soper, S. A., Spivak, D. A., “Surface immobilization methods for aptamer diagnostic applications”, Anal Bioanal Chem (2008) 390:1009-1021.
5. Lee, J., So, H., Jeon, E., Chang, H., Won, K., Kim, Y. H., “Aptamers as molecular recognition elements for electrical nanobiosensors”, Anal Bioanal Chem (2008) 390:1023-1032.
6. Pultar, J., Sauer, U., Domnanich, P., Preininger, C., “Aptamer-antibody on-chip sandwich immunoassay for detection of CRP in spiked serum”, Biosensors and Bioelectronics 24 (2009) 1456-1461.
7. Kim, Y. S., Niazi, J. H., Gu, M. B., “Specific detection of oxytetracycline using DNA aptamer-immobilized interdigitated array electrode chip”, Analytica Chimica Acta 634 (2009) 250-254.
8. Song, M., Zhang, Y., Li, T., Wang, Z., Yin, J., Wang, H., “Highly sensitive detection of human thrombin in serum by affinity capillary electrophoresis/laser-induced fluorescence polarization using aptamers as probes”, Journal of Chromatography A., 1216 (2009) 873-878.
9. Mir, M., Jenkins, A. T. A., Katakis, I., “Ultrasensitive detection based on an aptamer beacon electron transfer chain”, Electrochemistry Communications 10 (2008) 1533-1536.
10. Mok, W., Li, Y., “Recent Progress in Nucleic Acid Aptamer-Based Biosensors and Bioassays”, Sensors 2008, 8, 7050-7084.
11. Tombelli, S., Minunni, M., Mascini, M., “Analytical applications of aptamers”, Biosensors and Bioelectronics 20 (2005) 2424-2434.
12. Walther, C., Meyer, K., Rennert, R., Neundorf, I., “Quantum Dot#Carrier Peptide Conjugates Suitable for Imaging and Delivery Applications”, Bioconjugate Chemistry, 2008, 19 (12), 2346-2356.
13. Keefe, A. D., Cload, S. T., “SELEX with modified nucleotides”, Chemical Biology 2008, 12:448-456.
14. Jenison, R. D.; Gill, S. C.; Pardi, A.; Polisky, B., “High resolution molecular discrimination by RNA,” Science, 1994, 263, 1425-1429.
15. Stojanovic, M. N.; de Prada, P.; Landry, D. W., “Aptamer based folding fluorescent sensor for cocaine,” J. Am. Chem. Soc., 2001, 123, 4928-4931.
16. Huizenga, D. E. and J. W. Szostak, “A DNA aptamer that binds adenosine and ATP,” Biochemistry, 1995. 34(2): p. 656-665.
17. Famulok, M., “Molecular recognition of amino acids by RNA aptamers,” J. Am. Chem. Soc., 1994, 116, 1698-1706
18. Harada, K.; Frankel, A. D., “Identification of two novel arginine binding RNAs,” EMBO, 1995, 14, 5798-5811
19. Wang, Y.; Rando, R. R., “Specific binding of aminoglycoside antibiotics to RNA,” Chem. & Biol., 1995, 2, 281-290
20. Koizumi, M. and R. R. Breaker, “Molecular recognition of cAMP by an RNA aptamer,” Biochemistry, 2000. 39(30): p. 8983-8992.
21. Tang, Z., Mallikaratchy, P., Yang, R., Kim, Y., Zhu, Z., Wang, H., Tan, W., “Aptamer Switch Probe Based on Intramolecular Displacement”, J. Am. Chem. Soc., 2008, 130 (34), 11268-11269.
22. Urata, H., Nomura, K., Wada, S., Akagi, M., “Fluorescent-labeled single-strand ATP aptamer DNA: Chemo- and enantio-selectivity in sensing adenosine”, Biochemical and Biophysical Research Communications 360 (2007) 459-463.
23. Nutiu, R., Li, Y., “Aptamers with fluorescence-signalling properties”, Methods 37 (2005) 16-25.
24. Liu, J., Zehui, C. and Yi, L., “Functional Nucleic Acid Sensors,” Chem. Rev. (2009), 109:1948-1998.
25. Daubresse, C., Grandfils, C., Jerome, R., Teyssie, P., “Enzyme Immobilization in Nanoparticles Produced by Inverse Microemulsion Polymerization, Journal of Colloid and Interface Science 168, 222-229 (1994).
26. Clark, H., A., Kopelman, R., Tjalkens, R., Philbert, M. A., “Optical Nanosensors for Chemical Analysis inside Single Living Cells. 2. Sensors for pH and Calcium and the Intracellular Application of PEBBLE Sensors”, Anal. Chem., 1999, 71 (21), 4837-4843.
27. Poulsen, A. K., Scharff-Poulsen, A. M., Olsen, L. F., “Horseradish peroxidase embedded in polyacrylamide nanoparticles enables optical detection of reactive oxygen species”, Analytical Biochemistry 366 (2007) 29-36.
28. ISO Reference, “Particle size analysis—Photon correlation spectroscopy”, ISO 13321 (1996).
29. Berne, B. J. and Pecora, R., “Dynamic Light Scattering With Application to Chemistry”, Biology and Physics (General Publishing Company, Toronto, 1976).
30. Johnson, Jr. C. S. Jr. and Gabriel, D. A., “Laser Light Scattering”, (Dover, N.Y., 1994).
31. Bruggemeier, S., et al., “Use of protein-acrylamide copolymer hydrogels for measuring protein concentration and activity,” Analytical Biochemistry (2004) 329(2): 180.
32. Lu, Z., et al., “Antigen responsive hydrogels based on polymerizable antibody Fab fragment,” Macromolecular Bioscience (2003), 3 (6): 296.
33. Jackson, D., et al., “Free radical induced polymerization of synthetic peptides into polymeric immunogens,” Vaccine (1997) 15(15): 1697.
34. Monji, N., et al., “Activated, N-substituted acrylamide polymers for antibody coupling—application to a novel membrane-based assay,” Journal of Biomaterials Science—Polymer Edition (1994) 5(5):407.
35. Arenkov, P. et al., “Protein microchips: use for immunoassay and enzymatic reactions”, Analytical Biochemistry 2000 Feb. 15; 278(2): 123-31.
36. Weston, P. and Avrameas, S, “Proteins coupled to polyacrylamide beads using glutaraldehyde”. Biochemical and biophysical research communications, 1971 Dec. 17; 45(6): 1574-80.
37. Roizenblat, R., et al., “Nanobiolistic delivery of indicators to the living mouse retina,” J. Neuorsci. Methods (2006) 153(1):154.
38. Longmuir, K., et al., “Effective targeting of liposomes to liver and hepatocytes in vivo by incorporation of a Plasmodium amino acid sequence”, Pharmaceutical Research (2006) 23(4):759.
39. Mehier-Humbert, S., and Guy, R., “Physical methods for gene transfer: Improving the kinetics of gene delivery into cells”, Advanced Drug Delivery Reviews (2005) 57(5):733.
40. McAllister, D., et al., “Microfabricated needles for transdermal delivery of macromolecules and nanoparticles: Fabrication methods and transport studies”, PNAS (2003) 100 (24): 13755.
41. Srinivasan, J., et al., ADP-specific sensors enable universal assay of protein kinase activity. Chemistry & Biology, 2004. 11(4): p. 499-508.
42. Palm, C. et al., “Quantitatively determined uptake of cell-penetrating peptides in non-mammalian cells with an evaluation of degradation and antimicrobial effects,” Peptides (2006), 27:1710.
43. Betat, H. et al., “Aptamers that recognize the lipid moiety of antibiotic Moenomycin A,” Biological Chemistry (2003) 384:1497.
44. Nicholls, D. G., Ferguson, S. J. Bioenergetics (2002) 3.
45. Johnson, L. N. Bioch Soc T 2009, 37, 627-641.
46. Xu, Z.; Singh, N. J.; Lim, J.; Pan, J.; Kim, H. N.; Park, S.; Kim, K. S.; Yoon, J. J Am Chem Soc 2009, 131, 15528-15533.
47. Kunzelmann, S.; Webb, M. R. J Biol Chem 2009, 284, 33130-33138.
48. Imamura, H.; Nhat, K. P. H.; Togawa, H.; Saito, K.; lino, R.; Kato-Yamada, Y.; Nagai, T.; Noji, H. P Natl Acad Sc USA 2009, 106, 15651-15656.
49. Sassanfar, M.; Szostak, J. W. Nature 1993, 364, 550-553.
50. Huizenga, D. E.; Szostak, J. W. Biochemistry 1995, 34, 656-665.
51. Liu, J. W.; Cao, Z. H.; Lu, Y. Chem Rev 2009, 109, 1948-1998.
52. Sazani, P. L.; Larralde, R.; Szostak, J. W. J Am Chem Soc 2004, 126, 8370-8371.
53. Tang, Z. W.; Mallikaratchy, P.; Yang, R. H.; Kim, Y. M.; Zhu, Z.; Wang, H.; Tan, W. H. J Am Chem Soc 2008, 130, 11268.

1.-28. (canceled)

29. A nanobiosensor comprising an aptamer sequence incorporated within a porous polymer nanoparticle to provide a solid affinity matrix, said nanoparticle having average particle size<100 nm, preferably <50 nm, wherein the aptamer is a DNA or RNA sequence.

30. The nanobiosensor of claim 29, wherein the aptamer was selected from a library of modified nucleotides and comprises a fluorescence/quench pair.

31. The nanobiosensor of claim 29, wherein the polymer is selected from polyacrylamide, poly(methylmethacrylate), Polylactate Polyglycolate Block Copolymers, Chitosan/Alginate, poly(ethylene-vinyl acetate) copolymer, crosslinked polystyrene, poly(pyrrole), poly(L-lysine), poly(ethylene glycol diacrylate), and Polylactic acid and polyglycolic acid (PLGA) block copolymers.

32. The nanobiosensor of claim 29, wherein the aptamer sequence is modified by a molecular beacon structure.

33. The nanobiosensor of claim 29, wherein the aptamer sequence is modified by a radiologic imaging modality.

34. The nanobiosensor of claim 29, wherein the aptamer sequence is modified by a signalling domain.

35. The nanobiosensor of claim 29, wherein the aptamer sequence is modified by a fluorescence/quench pair.

36. The nanobiosensor of claim 29, further characterized by comprising a targeting sequence and/or other targeting agent.

37. A nanobiosensor comprising an aptamer sequence incorporated within a porous polyacrylamide nanoparticle to provide a solid affinity matrix, said nanoparticle having average particle size<50 nm, wherein the aptamer sequence is either
modified at the 3′ end with a PEG linker, a short hybridizing sequence complementary to the 5′ end of the aptamer sequence and one partner of a fluorescence/quench pair, and
modified at the 5′ end with the other partner of a fluorescence/quench pair
or
modified at the 5′ end with a PEG linker, a short hybridizing sequence complementary to the 3′ end of the aptamer sequence and one partner of a fluorescence/quench pair, and
modified at the 3′ end with the other partner of a fluorescence/quench pair.

38. A nanobiosensor comprising an aptamer sequence incorporated within a porous polyacrylamide nanoparticle to provide a solid affinity matrix, said nanoparticle having average particle size<50 nm, wherein the aptamer sequence is either
modified at the 3′ end with an extender sequence and one partner of a fluorescence/quench pair, and
modified at the 5′ end with a complementary extender sequence and the other partner of a fluorescence/quench pair,
or
modified at the 5′ end with an extender sequence and one partner of a fluorescence/quench pair, and
modified at the 3′ end with a complementary extender sequence and the other partner of a fluorescence/quench pair.

39. The nanobiosensor of claim 37, wherein the aptamer is a DNA sequence.

40. The nanobiosensor of claim 28, wherein the aptamer is any one of SEQ ID NO. 1-8, 10 or 12-14.

41. Method of detecting concentrations of target ligand comprising use of the nanobiosensor of claim 28.

42. The method of claim 41 wherein the nanobiosensor is used in a biopsy sample, organ slice, isolated perfused organ, organotypic culture or organ in situ.

43. The method of claim 41 wherein the nanobiosensor is used in blood, plasma, urine, cerebrospinal fluid, tissue extracts, cell extracts or other body fluids, environmental samples or production samples.

44. The method of claim 41, wherein the nanobiosensor is used in multiple-well arrays or similar multi-sample detection systems.

45. The method of claim 41, wherein the nanobiosensor is used to quantify small molecule concentration in specific tissues.

46. The method of claim 41, wherein the nanobiosensor is used for intracellular ligand detection in vivo.

47. A cell comprising the nanobiosensor of claim 28.

48. Method of affinity chromatography comprising use of the nanobiosensor of claim 28 as affinity chromatography matrix.
